(19) United States
(12) Patent Application Publication
(10) Pub. No.: US 2024/0425905 A1
(43) Pub. Date: Dec. 26, 2024

(54) METHOD AND KIT FOR DETECTING MICRORNA

(71) Applicant: Advanced Precision Medicine Limited, Hong Kong (CN)

(72) Inventors: Gang Lu, Hong Kong (CN); Wai Yee Chan, Hong Kong (CN); Xian Wei Su, Hong Kong (CN); Jingye Zhou, Hong Kong (CN); Quiang Liu, Hong Kong (CN)

(73) Assignee: Advanced Precision Medicine Limited, Kong Kong (CN)

(21) Appl. No.: 18/686,263

(22) PCT Filed: Aug. 25, 2022
(86) PCT No.: PCT/CN2022/114948

§ 371 Date: Apr. 23, 2024

(30) Foreign Application Priority Data
Aug. 25, 2021 (CN) 202110979088.6

(51) Int. Cl. C12Q 1/6825; C12N 15/10; C12Q 1/48; C12Q 1/6806; C12Q 1/686; C12Q 1/6876; G01N 21/64

(52) U.S. Cl. CPC C12Q 1/6825; C12N 15/1096; C12Q 1/485; C12Q 1/6806; C12Q 1/686; C12Q 1/6876; G01N 21/6486; C12Q 2600/178; G01N 2333/9125

(57) ABSTRACT

Provided is a method for detecting a microRNA. The method comprises: adding both (a) a polyadenylic acid tailing reaction system and (b) a reverse transcription reaction system to a sample to be detected, and subjecting the mixture to a one-step method by means of using a universal reverse transcription primer to obtain a reverse-transcribed cDNA product; and amplifying the product with a downstream universal primer and a specific upstream primer and detecting the amplified product with a universal fluorescence probe in a DNA amplification reaction and fluorescence detection system. Further provided is a kit based on the above-mentioned method. The provided method and kit have significantly higher sensitivity and specificity than traditional methods, can realize high-throughput sensitive detection with a small amount of samples, are convenient to operate and consume a short amount of time, and can detect batches of target microRNAs at low cost. Therefore, the provided method and kit are suitable for the screening of various biological samples, early diagnosis, companion diagnosis and prognosis evaluation of clinical diseases, etc.

### TECHNICAL FIELD

The present invention relates to methods for detecting microRNAs in a sample. More specifically, the present invention relates to methods for detecting microRNAs in samples using reverse transcription and amplification reactions and fluorescent probes.

### TECHNICAL BACKGROUND

MicroRNA (microRNA, miRNA) is a kind of endogenous small molecule non-coding RNA with a length of 18-24 nucleotides, which is ubiquitous in various organisms. The primary transcripts of miRNA (pri-miRNA) are processed in the nucleus by RNase III and the double-stranded RNA binding protein Pasha to miRNA precursors (pre-miRNAs) with a stem-loop structure of about 70 nucleotides, and pre-miRNAs are processed by exportin5 and transported to the cytoplasm, and is cut by another RNase III (Dicer) to form a double strand of about 22 nucleotides, one of which is a mature miRNA molecule. Mature miRNA molecules negatively regulate target mRNAs, thereby achieving transcriptional regulation of target genes. With the progress of research, it was found that miRNA and non-coding RNA family are abundantly expressed in biological fluids, involved in various pathophysiological processes, responding more directly to changes in physiological conditions, and can be used as effective biomarkers.

Circulating nucleic acids (CNAs) refer to free DNA and RNA in plasma or serum, and are extracellular free nucleic acids. Since Mandel and Meta is discovered circulating nucleic acids in plasma and serum in 1948, corresponding diagnostic techniques have emerged. For example, the discovery of fetal DNA in maternal plasma offers the potential for non-invasive prenatal diagnosis and monitoring of various pregnancy-related disorders. Similarly, tumor-derived circulating DNA has been demonstrated in a variety of cancer patients. Tumor-derived circulating RNA and fetal nucleic acid are also used in tumor and prenatal diagnosis. Because of this, scientists have paid more and more attention to the study of circulating miRNAs in recent years. Since the discovery of the significant significance of circulating miRNA in prenatal diagnosis, lung cancer, colon cancer, prostate cancer, diabetes, and drug-induced liver injury, people have been working on the non-invasive diagnosis and early warning of circulating miRNA for clinical diseases, applied research.

For miRNA detection methods, many detection and analysis methods have been reported, including RNA Northern blotting method, microarray chip method, electrochemical biosensor method and real-time fluorescent quantitative PCR method. The Northern blotting method has high specificity, but the method has low sensitivity and cumbersome steps, and is not suitable for high-throughput detection. Microarray chips can be used for rapid and high-throughput detection, but the detection method has poor reproducibility and accuracy. The detection of miRNA by electrochemical biosensor needs to be labeled with electrical activity, the operation steps are complicated, and the sensitivity is not high, and the detection of miRNA with low level expression is of poor effect.

Various miRNA detection technologies based on real-time fluorescent quantitative PCR (qPCR) have been developed in the field, but there are certain problems. Since the microRNA sequence is short, it cannot be directly amplified by qPCR, so it is generally extended during the microRNA reverse transcription reaction. The method of reverse transcription amplification detection of miRNA using stem-loop primers or miRNA tailing is currently the main method for identifying and synthesizing the first strand of microRNA Among them, the stem-loop method utilizes specific stem-loop primers to perform reverse transcription on microRNAs. Jung U et al. (A universal TaqMan-based RT-PCR protocol for cost-efficient detection of small noncoding RNA.RNA.2013 December; 19(12):1864-73.) disclose a method using stem-loop primers for reverse transcription of small RNA, and the use of universal probes for recognizing stem-loop sequences or specific probes for recognizing microRNA sequences. The stem-loop reverse transcription primer used in the method contains a specific sequence complementary to a specific microRNA and a longer universal sequence with a stem-loop structure. In the reverse transcription process, each reverse transcription process can only target a single microRNA, resulting in a requirement for a large sample and difficulty for high-throughput detection.

Another commonly used method of tailing is to use poly (A) polymerase to add a poly A tail to the 3′ end of the microRNA, and then use a primer containing poly T to carry out reverse transcription, so that the cDNA chain of the microRNA adds a section connector. The cDNA products of all microRNAs can be obtained by one reverse transcription, and the fluorescent quantitative PCR is further carried out. However, in the PCR process, custom probes need to be synthesized for each independent microRNA, resulting in high cost and difficulty in high-throughput detection. Kang K et al. (A novel real-time PCR assay of microRNAs using S-Poly(T), a specific oligo(dT)reverse transcription primer with excellent sensitivity and specificity. PLoS One. 2012; 7(11):e48536.) disclosed an improved method, using a two-step method to synthesize cDNA products from microRNA. The partial sequence of poly T and the specific reverse amplification primers of extended fragments were reverse transcribed. In this method, a universal TaqMan probe is used to identify partial sequences on the extended fragment. However, this method requires a two-step method of tailing and reverse transcription of the microRNA, which is cumbersome and time-consuming, and it is difficult to synthesize specific reverse amplification primers and perform a separate reverse transcription reaction for each microRNA; it is not suitable for volume detection.

Fluorescent quantitative PCR based on SYBR Green has been used in the field to detect the expression of free miRNA. However, SYBR Green has the defect of non-specific binding to double-stranded DNA and causing false positives, and cannot remove the fluorescent signal generated by non-specific amplification, resulting in inaccurate quantification. Busk PK (A tool for design of primers for microRNA-specific quantitative RT-qPCR. BMC Bioinformatics.2014 Jan. 28; 15: 29.) discloses a method to add a poly A tail to the 3′ end of the microRNA, then use the partial sequence of microRNA, as well as the downstream amplification primer of poly T and a Tag to carry out reverse transcription. The Busk PK method does not require custom probes, but it needs to synthesize amplification reverse primers for each independent microRNA. The detection method uses the fluorescence quantitative PCR method of SYBR Green, and the detection sensitivity and specificity are not enough, and it is difficult to carry out High-throughput detection.

US 20130045885A1 discloses a combination of stem-loop method and tailing method, which uses a two-step method to synthesize cDNA products from microRNAs. The first step is to add a poly A tail to the 3′ end of microRNAs for tailing, in the second step of the reaction, the microRNA is reverse-transcribed using a universal stem-loop primer with a poly T fragment, and the synthesized cDNA product is detected using a universal probe that recognizes the stem-loop sequence. The method disclosed in US 20130045885A1 adopts a combination of stem-loop universal reverse transcription primer and poly T sequence, it is complicated and time-consuming and has other issues.

Therefore, the existing methods for detecting miRNA in biological samples have the problems of cumbersome detection steps, complicated probe and/or primer design, high detection cost, or failure to meet the requirements of high-throughput detection.

There is a need in this field for a better method for detecting microRNAs, which is easy to operate, requires less sample size, has high sensitivity, good selectivity, rapid analysis, good overall cost-effectiveness, and can achieve the purpose of high-throughput detection.

### SUMMARY OF THE INVENTION

The invention provides a method for detecting microRNA (microRNA, miRNA), which comprises the following steps:
(a) adding a poly-adenylic acids (Poly (A)) tailing reaction system to the sample containing microRNA to be tested to obtain an RNA molecule with polyadenylic acids at the 3′ end;
the tailing reaction system contains: an enzyme for catalyzing the poly-adenylic acids tailing reaction, and a substrate for the tailing reaction;
(b) adding a reverse transcription reaction system to obtain a reverse transcribed cDNA product;
the catalytic system contains: an enzyme for catalyzing a reverse transcription reaction, a universal reverse transcription primer, and a substrate for reverse transcription reactions; wherein, the 3′ end of the universal reverse transcription primer contains a poly T oligonucleotides fragment, and the 5′ end is an extend tag sequence fragment;
(c) amplifying the obtained cDNA reverse transcription product in a DNA amplification reaction and fluorescence detection system, and detect the existence and/or quantity of the amplified product, so as to obtain the detection result of the microRNA. In one of the embodiments, the polymerase chain reaction (PCR) is used in step (c) to amplify the cDNA reverse transcription product obtained, and the fluorescent probe method is used to detect the microRNA by detecting the signal of the reporter group in the cyclic amplification of the polymerase chain reaction.

In another aspect of the present invention, the cDNA reverse transcription product obtained in step (c) is amplified by polymerase chain reaction (PCR) to obtain an amplified product, wherein the primer pair used in the amplification includes: a universal reverse primer (or “downstream primer” herein) and a specific forward primer (or “upstream primer” herein) that specifically recognize the target microRNA; at the same time, a universal fluorescent probe is added to the reaction system, wherein the 3′ and 5′ ends of the universal fluorescent probe are labeled with reporter group or quencher group. respectively, and detecting the signal of the reporter group in the cyclic amplification step of the polymerase chain reaction, thereby obtaining the detection result of the microRNA.

Wherein, the universal reverse primer specifically recognizes the sequence of the universal reverse transcription primer; the specific forward primer recognizes the sequence of the target microRNA; and the universal fluorescent probe can specifically hybridize with the amplified product of the universal reverse primer and the specific forward primer.

MicroRNA (microRNA, miRNA) is an RNA molecule ranging in size from 20 to 25nucleotides, and is one of the non-coding RNA family, miRNAs are processed through “hairpin precursors” and can act as negative regulators in gene expression, downregulating many genes MicroRNAs are first transcribed as long “primary-transcripts” (also referred to as pri-microRNAs). These “primary transcripts” are then shortened to about 70 nucleotides, resulting in so-called “stem-loop structures”, also known as “pre-miRNAs”. Pre-miRNAs are exported into the cytoplasm where they are further processed, thereby producing native miRNA molecules of approximately 22 nucleotides in length.

In the method of the present invention, when the target microRNA (or called target miRNA) exists in the sample, after the tailing reaction of (a) and the reverse transcription of (b), due to the use of a universal reverse transcription primer whose 3′ end contains a poly T oligonucleotide fragment, all microRNA templates in a sample can be obtained at one time, achieving the purpose of high throughput. In step (c), the miRNA library obtained in the previous step is amplified by adding specific forward primers for target microRNAs, and one or more target microRNAs can be amplified for the next step of detection. At the same time, the design of reverse primers can be simplified and the cost of detection applications can be reduced by using universal reverse amplification primers that recognize sequences in the aforementioned universal reverse transcription primer. When the target microRNA exists in the sample, amplicons including the sequence of the target microRNA and the sequence of the universal reverse transcription primer can be specifically obtained. At the same time, in step (c), the probe hybridizes with the amplicon comprising the target microRNA sequence and the universal reverse transcription primer sequence, and is hydrolyzed at each step of the cyclic amplification, whereby the reporter group falls off from the probe and leaves the quenching group, releasing a fluorescent signal, thereby providing the detection result of the microRNA. By using a universal fluorescent probe that recognizes and binds to the poly-T oligonucleotide fragment of the universal reverse transcription primer and one or more bases in the extend tag sequence fragment, the design of the probe can also be simplified and cost of the detection method can be reduced.

In one aspect of the present invention, the enzyme that catalyzes the poly-adenylic acids tailing reaction in the Poly (A) tailing reaction system in step (a) is an enzyme with polyadenylation activity. Many enzymes with polyadenylation activity are known to those skilled in the art. Polyadenylation activity is to use the 3′-end of ribonucleic acid as a substrate, and ribonucleotides can be added to the 3′-end in a suitable buffer, preferably adding at least 10-20 ribonucleosides acid. The polyadenylation active enzyme uses adenine-5′-triphosphate as a substrate. Enzymes with polyadenylation activity that can be used in the present invention include: Escherichia coli polyadenylate polymerase, yeast polyadenylate polymerase, bovine polyadenylate polymerase, frog polyadenylate polymerase, human polyadenylate polymerase Adenylate polymerase and plant polyadenylate polymerase, etc. In one aspect of the present invention, the enzyme having polyadenylation activity used in step (a) is Escherichia coli polyadenylate polymerase. In one aspect of the present invention, the Poly (A) tailing reaction system in step (a) includes a substrate for the tailing reaction, such as adenosine triphosphate (ATP). In one aspect of the present invention, the Poly (A) tailing reaction system in step (a) further includes a buffer component suitable for the enzyme reaction.

In one aspect of the present invention, the enzyme in the reverse transcription reaction system in step (b) is an enzyme with reverse transcriptase activity, which can be selected from viruses, bacteria and eukaryotic cells, especially enzymes from thermostable organisms. In the specification of the present invention, both the terms reverse transcription and reverse transcription can be used to indicate the process of synthesizing DNA using RNA as a template.

Enzymes with reverse transcriptase activity that can be used in the present invention include: HIV reverse transcriptase, M-MLV reverse transcriptase, EAIV reverse transcriptase, AMV reverse transcriptase, Thermus thermophilus DNA polymerase I, M-MLV RNA Enzyme H, Superscript, Superscript II, Superscript III, Sensiscript Reverse Transcriptase, ThermoScript and Thermo-X etc. In one aspect of the present invention, the reverse transcriptase used in step (b) is M-MLV reverse transcriptase.

In one aspect of the present invention, the reverse transcription reaction system in step (b) further includes buffer components suitable for reverse transcriptase reaction, including divalent ions, such as Mg²⁺ and Mn²⁺.

In one aspect of the present invention, the reverse transcription reaction system in step (b) further includes a reverse transcription primer. In the method of the present invention, the reverse transcription primer is a universal primer, that is, a primer that can be used for reverse transcribe various microRNAs in a sample to generate corresponding cDNA. The 3′ end of the universal reverse transcription primer has a poly-T oligonucleotides fragment, which can recognize the polyadenylated miRNA in step (a). In the present invention, the poly-T oligonucleotides fragment at the 3′ end of the universal reverse transcription primer has more than 10 bases, such as about 10-20 bases, preferably 15 bases. In the present invention, the 5′ end of the universal reverse transcription primer is an extend tag sequence fragment with more than about 20 bases (for example, 20-30 bases). In yet another aspect of the present invention. the 3′ end of the universal reverse transcription primer, that is, the upstream of the poly T oligonucleotide fragment, also includes an anchor base sequence for binding to the 5′ terminal of the poly (A) fragment. The anchor base sequence is, for example, VN, wherein “V” represents dATP, dGTP or dCTP; “N” represents any one of dATP, dTTP, dGTP or dCTP.

In one aspect of the present invention, steps (a) and (b) in the method are a one-step reaction, that is, adding the tailing reaction system and the reverse transcription reaction system to the microRNA-containing sample to be tested simultaneously to obtain a reverse-transcribed cDNA product.

Those skilled in the art can make the enzymes that catalyzes polyadenylate tailing reaction and reverse transcriptase activity to work in the same reaction system by adjusting the corresponding amount of various enzymes, suitable incubation temperature and incubation time; the enzymes exert their activities under the conditions and converts the microRNA in the sample to be tested into the corresponding cDNA.

In one aspect of the present invention, in step (c), various amplification methods known in the art can be used to amplify the obtained cDNA reverse transcription product, and detect the existence and/or quantity of the amplification product, so as to obtain the detection result of the microRNA.

In one of the embodiments of the present invention, in step (c), the polymerase chain reaction (PCR) is used to amplify the cDNA reverse transcription product obtained, and the fluorescent probe method is used to detect the microRNA by detecting the signal of the reporter group in the cyclic amplification of the polymerase chain reaction.

The term “PCR” or “polymerase chain reaction” refers to a reaction that uses a thermostable DNA polymerase to amplify a certain target nucleic acid molecule. The reaction system of the polymerase chain reaction includes DNA polymerase, and oligonucleotide primers (forward primer and reverse primer) that can specifically hybridize with the target nucleic acid, substrates for PCR amplification reactions such as deoxynuclear reaction buffer for nucleotide (dNTP) mixture, divalent ions such as Mg²⁺, etc. A “DNA polymerase” is an enzyme that catalyzes the polymerization of deoxynucleotides that will begin at the 3′-end of a primer that anneals to a polynucleotide template sequence and will continue toward the 5′-end of the template strand. Various DNA polymerase known in the art such as Taq DNA polymerase. Tth DNA polymerase, Pfu DNA polymerase, KOD DNA polymerase etc. all can be used in the present invention. The DNA molecules produced by a PCR reaction are called “amplification products”. “Primer” generally refers to a primer capable of serving as an initiation point for synthesis along a complementary strand under conditions that catalyze the synthesis of a primer extension product complementary to a nucleic acid strand (e.g., with a polymerase and a template with a complementary sequence, and at a suitable temperature). Oligonucleotides. In the polymerization reaction system, there can be one or more primers. The term “primer pair” refers to a set or pair of primers comprising a 5′ sense primer (sometimes referred to as “forward” or “upstream”) that hybridizes to the complement of the 5′ end of an amplified nucleic acid sequence and a 3′ antisense primer (sometimes referred to as “reverse” or “downstream”) hybridizes to the 3′ end of the amplified sequence.

In the present invention, in step (c), the universal reverse primer in the primer pair used for PCR amplification of the cDNA reverse transcription product obtained in the previous step recognizes the universal reverse transcription primer, that is, it has the all or some of the complementary sequence of the universal reverse transcription primer. In one aspect of the present invention, the universal reverse primer recognizes the extend tag sequence fragment of the universal reverse transcription primer, for example, has a sequence complementary to the 5′ end fragment of the extend tag sequence of the universal reverse transcription primer.

In addition, in step (c), the specific forward primer in the primer pair used for PCR amplification of the cDNA reverse transcription product obtained in the previous step specifically recognizes the full length or part of the target microRNA. Preferably, the specific forward primer has a sequence corresponding to the full length of the target miRNA. For example, the specific forward primer has the full-length sequence of the miRNA, wherein the base U in the miRNA is changed to T.

In one aspect of the present invention, the Tm value of the universal reverse primer in step (c) is about 55-65° C. preferably about 60° C.

In one aspect of the present invention, the Tm value of the specific forward primer in step (c) is about 55-65° C. preferably about 60° C.

In the method of the present invention, the selection of the universal reverse primer used in the present invention includes the selection of its length and base so that the Tm value of the primer is suitable for matching with the Tm value of the specific forward primer. The method provided by the present invention can be used to detect a large number of different miRNAs on the same sample, and the specific forward primer usually has a sequence corresponding to the full length of the target miRNA. The use of universal reverse amplification primers facilitates the simultaneous adaptation of a large number of specific forward primers and amplification reactions for different target miRNAs.

In the present invention, the universal fluorescent probe in step (c) can specifically hybridize to the amplification products of the aforementioned specific upstream primer and downstream universal primer.

In one aspect of the present invention, the universal fluorescent probe recognizes and binds to a fragment corresponding to the universal reverse transcription primer in the amplification product, that is, has a sequence complementary to all or part of the universal reverse transcription primer. For example, the probe recognizes and binds to one or more bases in the Poly (T) oligonucleotide fragment of the universal reverse transcription primer and the extend tag sequence fragment.

In one aspect of the present invention, the length of the universal fluorescent probe is about 15-30 bases, preferably 19-24 bases, such as 19 bases. In one aspect of the present invention, the 5′ end of the universal fluorescent probe has about 10-15 poly-adenylic acids, and the 3′ end has one or more bases complementary to the extend tag sequences of the universal reverse transcription primer.

In step (c) of the method of the present invention, fluorescent probes, or TaqMan detection probes, are used to detect PCR amplification products. A probe refers to a nucleic acid molecule that is complementary to a target sequence (such as a PCR amplification product) and capable of forming a hybrid. The 3′ and 5′ ends of the fluorescent probe are respectively labeled with a reporter group or a quencher group, and the signal of the reporter group can be quenched by the quencher group. The probe can specifically hybridize with the amplification products of the specific upstream primer and the downstream universal primer. After the probe recognizes and forms a DNA double strand with the amplification product, it can be hydrolyzed by the 5′-3′ exonuclease activity of DNA polymerase, thereby releasing the reporter group and the quencher group.

The term “reporter group” generally refers to a moiety that produces a detectable emission of fluorescent or luminescent radiation that can be transferred to a suitable FRET quencher in sufficient proximity. Typically, such molecules are dyes. The term “quencher” generally refers to a moiety that reduces and/or is capable of reducing the emission of detectable fluorescent or luminescent radiation. The quencher molecule results in a decrease in fluorescence emission from the reporter, whereby the reporter/quencher forms a FRET pair. The term “FRET” (fluorescence resonance energy transfer or Förster type resonance energy transfer) generally refers to a dynamic distance-dependent interaction between the electronic states of two dye molecules.

In one aspect of the present invention, the 3′ and 5′ ends of the universal fluorescent probe in step (c) are respectively labeled with a reporter group or a quencher group. In one aspect of the present invention, the 3′ end of the probe is labeled with a quencher group and the 5′ end is labeled with a reporter group.

In one aspect of the present invention, the reporter group is a fluorescent reporter group. In one aspect of the present invention, the reporter group is selected from FAM, VIC, HEX, TET, TAMRA, etc., preferably FAM.

In one aspect of the present invention, wherein the quenching group is selected from BHQ1, BHQ2, BHQ3, MGB, etc., preferably MGB.

In one aspect of the present invention, the above method for detecting microRNA is used for in vitro detection.

In one aspect of the present invention, the above-mentioned method for detecting microRNA in vitro is used for non-diagnostic and non-therapeutic applications.

The present invention also provides the use of reagents to prepare a kit for implementing the method for detecting microRNA of the present invention. Wherein, the reagents include the reagents of the following aforementioned reaction system: (a) poly-adenylic acids tailing reaction system, which contains: an enzyme for catalyzing the poly-adenylic acids tailing reaction, and a substrate for the tailing reaction;
(b) a reverse transcription reaction system, which contains: an enzyme for catalyzing a reverse transcription reaction, a universal reverse transcription primer; and
(c) PCR amplification reaction and fluorescence detection system.

The enzyme used to catalyze the poly-adenylic acids tailing reaction, the substrates used for the tailing reaction; the enzyme used to catalyze reverse transcription reaction, the general reverse transcription primer; and the enzyme used to catalyze polymerase chains reactions, substrates for PCR amplification reactions, the downstream universal primer, the specific upstream primer that specifically recognize target microRNAs, and the universal fluorescent probe in each said system are as previously described and defined.

The present invention also provides kits for carrying out the aforementioned methods of the present invention. The test kit provided by the invention comprises:
(a) poly-adenylic acids (Poly (A)) tailing reaction system, which contains: an enzyme for catalyzing the Poly (A) tailing reaction, and a substrate for the tailing reaction;
(b) a reverse transcription reaction system, which contains: an enzyme for catalyzing a reverse transcription reaction, and a universal reverse transcription primer;
(c) PCR amplification reaction and fluorescence detection system, which contains: an enzyme for catalyzing the polymerase chain reaction, substrates for PCR amplification reactions, a universal reverse primer and a specific forward primer that specifically recognize a target microRNA, a universal fluorescent probe.

The enzyme used to catalyze the poly-adenylic acids tailing reaction, the substrates used for the tailing reaction; the enzyme used to catalyze reverse transcription reaction, the general reverse transcription primer; and the enzyme used to catalyze polymerase chains reactions, substrates for PCR amplification reactions, the downstream universal primer, the specific upstream primer that specifically recognize target microRNAs, and the universal fluorescent probe in each said system are as previously described and defined.

In one aspect of the present invention, the Poly (A) tailing reaction system and the reverse transcription reaction system in the kit are configured to be in one reaction system. The method and the kit of the present invention are suitable for carrying out the polyadenylic acid tailing reaction and the reverse transcription reaction in one step, that is, the tailing reaction and the reverse transcription reaction are carried out simultaneously in one reaction system to obtain a reverse-transcribed cDNA product. In the combined Poly (A) tailing reaction system and reverse transcription reaction system, the enzymes and substrates required for the Poly (A) tailing reaction and reverse transcription reaction are included, and by adjusting the corresponding amount of various enzymes, ion concentration in the buffer and other conditions, make the enzymes that catalyze the polyadenylic acid tailing reaction and the reverse transcriptase exert their activity under the same reaction system and solution conditions. Thus, in one aspect of the present invention, a method for detecting microRNA is provided, comprising the following steps:

A method for detecting microRNA, comprising the steps of:

Step 1: adding the following at the same time to the test sample containing microRNA:
(a) a poly-adenylic acids tailing reaction system for obtaining RNA molecules with poly adenylic acids at the 3′ end, the poly-adenylic acids tailing reaction system contains: an enzyme for catalyzing a poly-adenylic acids tailing reaction, and a substrate for the tailing reaction; and (b) a reverse transcription reaction system for obtaining a reverse-transcribed cDNA product; the reverse transcription system contains: an enzyme for catalyzing the reverse transcription reaction, and a universal reverse transcription primer; wherein, the 3′ end of the universal reverse transcription primer contains a poly T oligonucleotides fragment, and the 5′ end contains an extend tag sequence fragment;

optionally, the Poly (T) oligonucleotides fragment at the 3′ end of the universal reverse transcription primer contains about 10-20 bases, and the extended tag sequence fragment at the 5′ end contains about 20-30 bases;

Step 2: carrying out polymerase chain reaction (PCR) with the cDNA reverse transcription product obtained in Step 1 in a DNA amplification reaction and fluorescence detection system to obtain an amplification product, wherein the primer pair used in the amplification includes: a universal reverse primer and a specific forward primer specifically recognizing a target microRNA; at the same time, adding a universal fluorescent probe to the reaction system, and detecting the signal of the reporter group in the cyclic amplification step of the polymerase chain reaction, so as to obtain the detection result of the microRNA.

optionally, the universal reverse primer specifically recognizes the sequence of the universal reverse transcription primer; optionally, the specific upstream primer specifically recognizes the full length or part of the target microRNA; and optionally, the universal fluorescent probe can specifically hybridize with the amplification products of the specific upstream primer and the downstream universal primer, preferably, its length is about 19-24 bases, for example, the 5′ end has about 10-15 oligonucleotides polyadenylic acid; and for example, its 3′ end also has a plurality of bases complementary to the extended tag sequence fragment of the universal reverse transcription primer.

The method and kit of the present invention can be used to detect the target miRNA in the sample very efficiently and accurately. Specifically, the method of the present invention carries out miRNA Poly (A) tailing and reverse transcription simultaneously through a one-step approach, uses the universal reverse transcription primer sequence, adding Poly (A) tails to all miRNAs through one reverse transcription reaction and completes the PCR template preparation of all miRNAs; then by using the universal fluorescent probe for the detection of all miRNAs after reverse transcription, in which specific target miRNAs can be specifically detected by a universal reverse amplification primer and a specific forward amplification primer.

The method provided by the present invention overcomes the defects of the detection methods in the prior art, and has the following advantages: 1. Obtain all target miRNA reverse transcription products with a small amount of samples quickly; 2. Improve the sensitivity and specificity of detection by designing a universal fluorescent probe according to the sequence of miRNA after Poly (A) tailing, which can distinguish the interfering sequence and the target sequence with only one single base difference; 3. Based on the design of miRNA-based rapid reverse transcription products and universal fluorescent probes, miRNA in samples can be detected in a high-throughput way, thus solves the problems of using customized TaqMan probes for miRNA detection, including the high cost, time-consuming, and the need to synthesize cDNA of each single miRNA issues. The method provided by the present invention has high sensitivity, good selectivity, and low requirement for detection samples. For example, 1 ml of sample can be used to screen as many as 500-1000 kinds of free microRNAs, which is especially suitable for detection of small amounts of samples and small amounts of target RNAs, such as screening of circulating microRNAs in clinical samples. The present invention provides a platform for the processing of biological samples, the acquisition of large amounts of miRNAs, and the high-throughput detection of target miRNAs using PCR chips.

In the present invention, the terms have general definitions in the fields of biotechnology, organic chemistry, inorganic chemistry, etc. Specifically, relevant terms may be described and defined as follows.

The term “sample” refers to any substance that contains or is supposed to contain the microRNA of interest Examples include tissue or fluid samples isolated from individuals, including, but not limited to, for example, samples of skin, blood, spinal fluid, lymph, synovial fluid, urine, tears, blood cells, organs, tumors, and in vitro cell culture components. Preferably, the sample is plasma, serum, lymph, urine, saliva, milk, semen, vaginal fluid, tear fluid, spinal fluid and other body fluid samples. The individual may be a plant, an animal, a bacterium, or the like.

The term “target microRNA” or “target nucleic acid sequence” refers to a microRNA to be detected or a nucleic acid sequence to be amplified. A target sequence generally has a sequence complementary to a primer or probe, or is a sequence located between the sequences of two primers used for amplification.

As used herein, the term “Ct value (threshold cycle)” refers to the cycle number of real-time PCR used for real-time measurement of amplification products in the exponential phase range, when the measured fluorescence intensity increases significantly much higher than the baseline level. It gives information of the amount of initial template of the target RNA.

### BRIEF DESCRIPTION OF THE DRAWINGS

FIG. 1 is a schematic flowchart of an exemplary method for detecting microRNAs of the present invention.

FIG. 2 is a diagram of the experimental results of detecting nematode cel-miR-39 mimics (cel-miR-39 mimic) of different concentration gradients using an exemplary one-step reverse transcription and real-time fluorescent quantitative PCR detection microRNA method of the present invention. FIG. 2A shows the amplification curves of cel-miR-39 in six different concentration gradients. FIG. 2B shows the standard curve.

FIG. 3 is a graph showing results by using exemplary method of the present invention and SYBR Green method, respectively. FIG. 3A is a graph showing the results of sample detection using the SYBR Green method. FIG. 3B is a graph showing the results of sample detection by the method of the present invention.

FIG. 4 is a graph showing the detection results of samples using probes with different sequences in an exemplary method of the present invention.

FIG. 5 is a graph showing the experimental results of a method for detecting microRNAs using an exemplary one-step reverse transcription method of the present invention and real-time fluorescent quantitative PCR to detect the reverse transcription of nematode cel-miR-39 mimics (cel-miR-39 mimic) with different concentration gradients.

FIG. 6 is a comparative diagram of a method for detecting microRNA using the SYBR Green method. FIG. 6A is a graph showing the experimental results of cDNA templates obtained after reverse transcription of nematode cel-miR-39 mimic (cel-miR-39 mimic) with different concentration gradients. FIG. 6B is the dissolution curve of the Concentration 4 sample. FIG. 6C is the dissolution curve of the Concentration 5 sample.

FIG. 7 is a graph showing the results of detecting a large number of microRNAs in a sample using an exemplary method of the present invention.

FIG. 8 is a result diagram of the influence of DNA contamination on detection when the exemplary method of the present invention and the SYBR Green method are respectively used for sample detection. FIG. 8A is a graph showing the results of sample detection by the method of the present invention. FIG. 8B is a graph showing the results of sample detection using the SYBR Green method.

FIG. 9 is a graph showing the results of specificity of sample detection using an exemplary method of the present invention. FIG. 9A is a graph showing the results of amplifying let-7a-5p and let-7c-5p standard products (there is one base difference between the two) using let-7a-5p specific primers. FIG. 9B is a graph showing the result of amplifying let-7a-5p and let-7c-5p standards with let-7c-5p specific primers.

### DETAILED DESCRIPTION OF THE INVENTION

The invention is further illustrated by the following examples, but these examples do not limit the invention in any respect.

### Example 1 Extraction, Detection and Quantification of Free MiRNA in the Sample Solution

Experimental equipment and reagents:

PCR instrument (ABI Veriti), fluorescent quantitative PCR (ABI QuantStudio™ 6), tube (Axygen #PCR-0208-C), fluorescent quantitative 96-well PCR plate (ABI #AB0600). low-temperature high-speed centrifuge (Eppendorf 5424R), EP tube, pipette man (Eppendorf), miR-Prep® General nucleic acid extraction kit (GeneDotech #GD-101, Shenzhen Jingdu Medical Instrument Technology Co., Ltd.), nematode cel-miR-39 mimic (Shanghai Aibosi Biotechnology Co., Ltd.), miR-Prep® One-step reverse transcription general kit (GeneDotech #GD-102, Shenzhen Jingdu Medical Equipment Technology Co., Ltd.), Fluorescent quantitative PCR reagent (Takara #RR390A).

The following primers and probes were synthesized and provided by Shanghai Aibosi Biotechnology Co., Ltd.

Primers and probes:

Universal reverse transcription primer:

(SEQ ID NO. 1)
5′GTCCGAGCAGCACGATCCGGTGACCAGTTTTTTTTTTTTTTTVN 3′
(V: A C G, N: ACGT)

Universal reverse primer:

(SEQ ID NO. 2)
5′GTCCGAGCAGCACGATC 3′

cel-miR-39 specific forward primer:

(SEQ ID NO. 3)
5′CACCGGGTGTAAATCAGCTTG 3′

Universal fluorescent probe:

(SEQ ID NO. 4)
FAM-5′-AAAAAAAAAAAAAAACTGG-3′-MGB

Experimental steps:
a) Collection of plasma samples

Collect normal human peripheral blood plasma samples from volunteers, use EDTA anticoagulant tubes to collect 3 ml of peripheral blood, place at room temperature for 1 h, centrifuge at 1600 g for 10 min, absorb the upper layer of plasma, divide it into 200 μl tubes, and store it in a −80° C. refrigerator.
b) Plasma free miRNA extraction

Take 200 μl of fresh plasma, according to the instructions of the miRNA extraction kit, add 800 μl of lysate to every 200 μl of plasma, invert and mix 3-5 times, add serially diluted exogenous reference substance cel-miR-39 mimic, and 10 μl of auxiliary extraction agent (50 ng/μl tRNA), add 200 μl chloroform to the EP tube. Vortex vigorously for 15 s to mix. At room temperature (15-25° C.), place the EP tube on the bench for 2-3 minutes. Centrifuge at 12,000 g for 15 minutes at 4° C. After centrifugation, the sample was separated into 3 layers: the upper layer was a colorless aqueous layer containing RNA, the middle white layer containing protein, and the lower red organic layer. Transfer the upper aqueous phase (˜500 μl) to a new EP tube. Add an equal volume of isopropanol and mix thoroughly by pipetting up and down several times. Place at room temperature for 10-15 min. Transfer 500 μl of the mixture to an RNase-free adsorption column, centrifuge at 8,000 g for 30 seconds, discard the effluent, then transfer the remaining mixture to an RNase-free adsorption column, centrifuge at 8,000 g for 30 seconds, Discard the flow-through. Add 750 μl of washing solution with 75% ethanol to the RNase-free adsorption column, centrifuge at 8,000 g for 30 seconds, and pour off the supernatant. Add 500 μl of 75% ethanol at 8,000 g, centrifuge for 2 minutes, pour off the supernatant, add 15 μl of nuclease-free water to the adsorption column, and let stand at room temperature for 1 minute. Centrifuge at 12,000 g for 1 minute. discard the adsorption column.
c) One-step reverse transcription and real-time fluorescent quantitative PCR of miRNA:

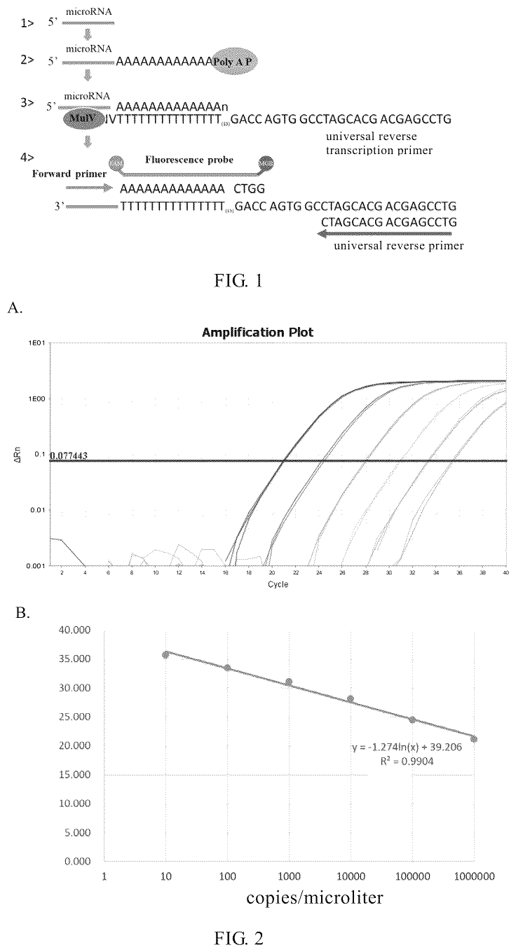

The flowchart of the exemplary miRNA detection method of the present invention used in this Example is shown in FIG. 1. The method includes: Step 1: adding a Poly (A) tailing reaction system to the sample containing microRNA to obtain an RNA with polyadenylic acids (polyp-A) at the 3′ end. Step 2: adding a reverse transcription reaction system to obtain a reverse transcribed cDNA product, wherein the 3′ end of the universal reverse transcription primer is an oligo (dT) fragment of 10-20 bases, and the 5′ end is an extend tag sequence fragment with more than about 20 bases; Step 3: carrying out polymerase chain reaction (PCR) with the cDNA reverse transcription product to generate amplicons, wherein the primer pair comprises: the downstream universal amplification primer that recognizes and binds the universal reverse transcription primer and the specificity upstream amplification primer that recognizes microRNA sequence; Step 4: Add a universal fluorescent probe to the PCR reaction system, the probe recognizes and binds to one or more bases in the oligo (dT) fragment of the universal reverse transcription primer and the extend tag sequence fragment, and the signal from the fluorescent reporter is detected during each cyclic amplification step.

In the preferred technical solution given in this example, steps 1 and 2 are carried out in a one-step approach, that is, the Poly (A) tailing reaction and reverse transcription reaction are carried out in one single reaction system: adding reagents of the tailing reaction system and the reverse transcription reaction system into the microRNA-containing sample at the same time, so as to simultaneously perform the tailing reaction and the reverse transcription reaction, thereby forming a reverse-transcribed cDNA product. The method for detecting miRNA provided by the present invention using the universal reverse transcription primer, the specific forward amplification primer and the universal downstream amplification primer, and the universal fluorescent probe as mentioned herein is also referred as the IntelliMiR fluorescent probe method.

Specifically, the method steps in this Example include:

One-step Poly (A) tailing and reverse transcription:

Take 5 μl of total RNA, and add 5 μl of nucleic acid-free water to dilute it, add to a tube, add 1 μl of M-MLV reverse transcriptase and 0.4 μl of polymerase A, and then add 8.6 μl of reverse transcription mixture (working concentration: dNTP 1 mM, ATP 0.1 mM, 250 mM NaCl, 50 mM Tris-HCl 10 mM MgCl2, universal reverse transcription primer 1 μM), after mixing, use a pipette to mix 3-5 times, put the tube into the PCR machine, setting program: 60 min at 42° C., 5 min at 95° C., 5 min at 4° C.

Probe method real-time fluorescent quantitative PCR:

The 10 μl fluorescent quantitative reaction system includes: 5 μl of 2×PCR reaction solution (including Taq enzyme, dNTP mixture, MgCl₂, etc.), 0.5 μl of cel-miR-39 forward amplification primer (10 μM), 0.5 μl of reverse universal primer (10 μM), miRNA cDNA (10-fold diluted) 1 μl, universal fluorescent probe (10 μM) 0.5 μl, ROX Dye 0.2 μl, nuclease-free water 2.3 μl.

Fluorescence quantitative PCR instrument reaction program: 95° C. for 30 s, 40 cycles (95° C. for 5 s, 60° C. for 30 s), each sample reaction contains 3 duplicates.

Test sample:

Serial concentration dilution of different samples: the nematode cel-miR-39 mimic (the amount of 1 mol microRNA=6.02×10²³ copies), and diluted to different concentrations 10⁶, 10⁵, 10⁴, 10³, 10², 10 copies/microliter, were added to 200 μl of human plasma, and RNA was extracted according to the miRNA extraction procedure described in Example 1.

FIG. 2 is a diagram of the experimental results of detecting nematode cel-miR-39 mimics (cel-miR-39 mimic) of different concentration gradients using an exemplary one-step reverse transcription and real-time fluorescent quantitative PCR microRNA detection method of the present invention (the IntelliMiR fluorescent probe method). FIG. 2A shows the amplification curves of cel-miR-39 in six different concentration gradients. FIG. 2B shows the standard curve. According to the Ct values obtained by 6 concentrations of cel-miR-39, a fitting curve and the formula y=1.274 ln (x)+39.206 were obtained, the coefficient of determination R2=0.9904.

It can be seen that the fluorescent probe method provided by the present invention can be used for the detection of miRNA in the sample solution. With the dilution of different concentrations of standard products, a standard curve can be established to accurately quantify the content of miRNA in the sample solution.

### Example 2 Comparing the Detection of MiRNA by the Fluorescent Probe Quantitative Method of the Present Invention and the SYBR Green Method

Plasma processing and miRNA extraction were carried out according to the method and steps described in Example 1, wherein the samples tested included:

Test sample 1: 200 μl plasma+10³ copies of cel-miR-39 mimic/μl+co-extraction agent;

Test sample 2: 200 μl plasma+10³ copies of cel-miR-39 mimic/μl;

Test sample 3: 200 μl plasma.

The probe fluorescence quantification method was carried out as described in Example 1.

The method and steps of using SYBR Green fluorescent quantitative PCR for comparison are as follows:

10 μl fluorescence quantitative reaction system includes: 2×PCR reaction solution (including Taq enzyme, dNTP mixture, MgCl₂, SYBR Green I fluorescent dye) 5 μl, cel-miR-39 upstream specific primer (10 μM) 0.5 μl, universal downstream primer (10 μM) 0.5 μl, miRNA cDNA product (10-fold diluted product) 1 μl, ROX Dye 0.2 μl, nuclease-free water 2.8 μl.

Fluorescence quantitative PCR instrument reaction program: 95° C. for 30 s, 40 cycles (95° C. for 5 s, 60° C. for 30 s), each sample reaction set up 3 duplicate wells.

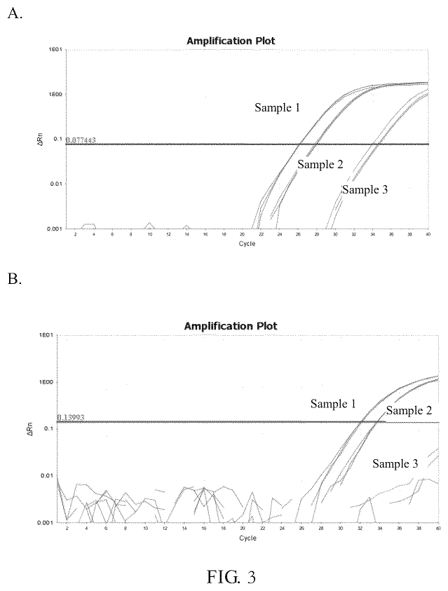

FIG. 3 is a graph showing results by using exemplary IntelliMiR fluorescent probe method of the present invention and SYBR Green method, respectively. FIG. 3A is a graph showing the results of sample detection using the SYBR Green method. FIG. 3B is a graph showing the results of sample detection by the IntelliMiR fluorescent probe method of the present invention.

As shown in FIG. 3A, the SYBR Green method was used to detect three samples and obtain amplification curves. Sample 3 does not contain exogenous cel-miR-39, and the amplification curve still appears, indicating that non-specific amplification occurs when the SYBR Green method is used to detect the expression of liquid miRNA.

As shown in FIG. 3B, the IntelliMIR fluorescent probe method was used to detect the three samples, and the amplification curves were obtained. There is no obvious amplification curve in sample 3, indicating that the method of the present invention does not produce non-specific amplification when detecting the expression level of miRNA. The results showed that the Ct value in sample 1 was significantly smaller than that in sample 2, that is, the amount of cel-miR-39 detected in sample 1 was greater than that in sample 2, which accurately reflected that the addition of co-extraction agents during the solution miRNA extraction process could significantly increase the miRNA extraction yield.

It can be seen that the real-time fluorescent quantitative PCR method provided by the present invention can specifically distinguish the miRNA in the solution sample and carry out quantitative determination.

### Example 3 Detect MiRNA in the Sample Solution Using Different Fluorescent Probes

Universal fluorescent probes with different sequences are used to detect miRNA in samples. The plasma sample processing, miRNA extraction and reverse transcription methods were carried out according to Example 1.

Probe 1:

(SEQ ID NO. 4)
FAM-5′-AAAAAAAAAAAAAAACTGG-3′-MGB

Probe 2:

(SEQ ID NO. 5)
FAM-5′-AAAAAAAAAAAAAAACTGGTCA-3′-MGB

Probe 3:

(SEQ ID NO. 6)
FAM-5′-AAAAAAAAAAACTGGTCA-3′-MGB

Probe 4:

(SEQ ID NO. 7)
FAM-5′-AAAAAAAAAAACTGGTCACC-3′-MGB

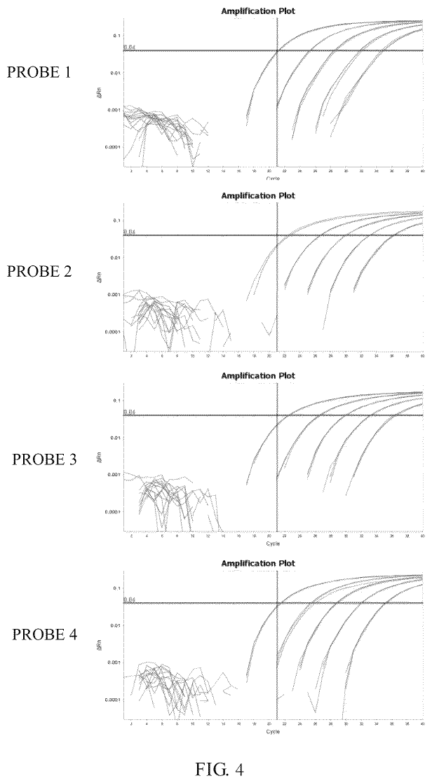

Sample: the serial dilution concentration of cel-miR-39 added to the detection sample is 10⁶, 10⁵, 10⁴, 10³, 10², 10 copies/microliter. FIG. 4 is a graph showing the detection results of samples using probes with different sequences in an exemplary method of the present invention. The results showed that the probes of different sequences tested in this experiment can be used to detect miRNA in the sample; the Ct value detected by the probe sequence 1 for the standard substance of the same concentration is the smallest, and the detection sensitivity is the highest.

### Example 4 Repeatability and Sensitivity of the Fluorescent Probe Quantitative Method of the Present Invention, and the Comparison with SYBR Green Method

Experimental equipment and reagents:

PCR instrument (Bro-rad #T100), fluorescent quantitative PCR (Lightcycler480 II), tube (Axygen #PCR-0208-C), fluorescent quantitative 96-well plate PCR (Roche #047296922001), low-temperature high-speed centrifuge (Eppendorf 5424R), EP tube, pipette (Eppendorf), nematode cel-miR-39 mimic (cel-miR-39 mimic, Shanghai Aibosi Biotechnology Co., Ltd.), miR-Prep® One-step reverse transcription general kit (GeneDotech #GD-102, Shenzhen Jingdu Medical Equipment Technology Co., Ltd.), Fluorescent quantitative PCR reagent (Takara #RR390A).

Primers and probes were synthesized and provided by Shanghai Aibosi Biotechnology Co., Ltd. The sequences thereof are the same as that in Example 1.

Experimental procedure: one-step tailing and reverse transcription are the same as disclosed in Example 1.

Test sample: cel-miR-39 mimic (the amount of 1 mol microRNA=6.02×10²³ copies), dilute and dissolve it into a standard template of 5 fmol/μl, take 5 μl of the standard template, reverse transcribe according to the one-step tailing reverse transcription system in Example 1 to obtain 20 μl of cDNA product, and then serially diluted to obtain a series of standard templates with concentration gradients: 1.25×10⁷, 1.25×10⁶, 1.25×10⁵, 1.25×10⁴, 6.125×10³, 3.06×10³, 1.5×10³, 7.5×10² copies/μl.

IntelliMiR fluorescent probe method real-time quantitative PCR:

10 μl fluorescence quantitative reaction system includes: 2×PCR reaction solution (including Taq enzyme, dNTP mixture, MgCl₂, etc.) 5 μl, cel-miR-39 upstream amplification primer (10 μM) 0.5 μl, downstream universal primer (10 μM) 0.5 μl, miRNA cDNA gradient dilution template (10-fold diluted product) 1 μl, universal fluorescent probe (10 μM) 0.5 μl, nuclease-free water 2.5 μl.

Fluorescent quantitative PCR instrument reaction program: The reaction was carried out on Lightcycler480 II fluorescent quantitative PCR instrument, the reaction program was 95° C. for 30 s, 40 cycles (95° C. for 5 s. 60° C. for 30 s), and 3 replicate wells were set for each sample reaction.

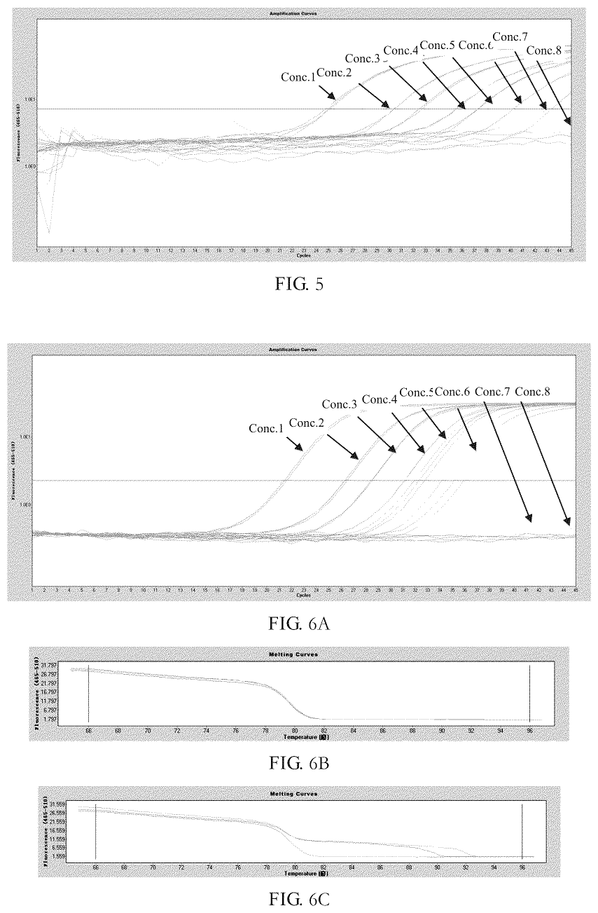

The experimental results are shown in FIG. 5. It can be seen that the amplification curve is S-shaped, which can detect different concentrations of standards of cel-miR-39mimics by fluorescent quantitative PCR, and its sensitivity can be as low as concentration of 7 (1.5×10³ copies).

### SYBR Green Method

Experimental procedure: one-step tailing and reverse transcription are the same as those in Example 1.

Test sample: cel-miR-39, dilute and dissolve it into a standard template of 5 fmol/μl, take 5 μl of the standard template, reverse transcribe according to the one-step tailing reverse transcription system in Example 1 to obtain 20 μl of cDNA product, and then serially diluted to obtain a series of standard templates with concentration gradients: 1.25×10⁷, 1.25×10⁶, 1.25×10⁵, 1.25×10⁴, 6.125×10³, 3.06×10³, 1.5×10³, 7.5×10² copies/μl. SYBR Green real-time fluorescent quantitative PCR:

10 μl fluorescence quantitative reaction system includes: 2×PCR reaction solution (including Taq enzyme, SYBR Green I fluorescent dye, dNTP mixture, MgCl 2, etc.) 5μl, cel-miR-39 upstream amplification primer (10 μM) 0.5 μl, downstream universal primer (10 μM) 0.5 μl, miRNA cDNA gradient dilution template (10-fold diluted product) 1 μl, nuclease-free water 3 μl.

Fluorescent quantitative PCR instrument reaction program: The reaction was carried out on Lightcycler480 II fluorescent quantitative PCR instrument, the reaction program was 95° C. for 30 s, 40 cycles (95° C. for 5 s, 60° C. for 30 s), and 3 replicate wells were set for each sample reaction.

The experimental results are shown in FIG. 6A. It can be seen that the amplification curve is S-shaped, which can realize the detection of different concentrations of standards of cel-miR-39 mimics by fluorescent quantitative PCR, and its sensitivity can reach concentration 4 (1.25×10⁴ copies). The amplification curves of replicate wells at concentration 5 (6.125×10³ copies) showed inconsistent, indicating that the detection sensitivity of this method in concentration 5 was not good. Further by analyzing the melting curve of the PCR product, as shown in FIG. 6B, the melting curve of concentration 4 (1.25×10⁴ copies) is a specific peak pattern. As shown in FIG. 6C, the melting curve of concentration 5 (6.125×10³ copies) appears non-specific profile, indicating non-specific amplification during amplification, resulting in inaccurate quantitation.

### Example 5 The Fluorescent Probe Quantitative Method of the Present Invention Detects Plural MicroRNAs in One Sample

Experimental steps: normal human plasma sample extraction, one-step tailing and reverse transcription conditions and steps are the same as those described in Example 1. Take 10 μl of cDNA product after reverse transcription of microRNA, add 50 μl of nuclease-free water to dilute and use in the next step Fluorescent quantitative PCR reaction.

IntelliMiR fluorescent probe method real-time quantitative PCR:

10 μl fluorescent quantitative reaction system includes: 5 μl of 2×PCR reaction solution (including Taq enzyme, dNTP mixture, MgCl₂, etc.), 0.5 μl of microRNA upstream amplification primer (10 μM), 0.5 μl of downstream universal primer (10 μM), miRNA cDNA dilution Template 1 μl, universal fluorescent probe (10 μM) 0.5 μl, nuclease-free water 2.5 μl.

Fluorescent quantitative PCR instrument reaction program: The reaction was performed on a Lightcycler480 II fluorescent quantitative PCR instrument. The reaction program was 95° C. for 30 s, 40 cycles (95° C. for 5 s, 60° C. for 30 s).

The names of more than 50 microRNAs and the forward amplification primer sequences used to detect them are as follows:

hsa-miR-9-5p forward primer:
CGCAGTCTTTGGTTATCTAGCTGTATGA
hsa-miR-128-3p forward primer:
TCACAGTGAACCGGTCTCTTT
hsa-miR-129-2-3p forward primer:
AAGCCCTTACCCCAAAAAAGCAT
hsa-miR-132-3p forward primer:
TAACAGTCTACAGCCATGGTCG
hsa-miR-149-5p forward primer:
TCTGGCTCCGTGTCTTCACTCCC
hsa-miR-181b-5p forward primer:
AACATTCATTGCTGTCGGTGGGT
hsa-miR-181d-5p forward primer:
AACATTCATTGTTGTCGGTGGG
hsa-miR-212-5p forward primer:
ACCTTGGCTCTAGACTGCTTACT
bsa-miR-491 forward primer:
AGTGGGGAACCCTTCCATGAGG
hsa-miR-598-3p forward primer:
TACGTCATCGTTGTCATCGTCA
hsa-miR-181a-5p forward primer:
AACATTCAACGCTGTCGGTGAGT
bsa-miR-195-5p forward primer:
CAGTAGCAGCAGAAATATTGGC
hsa-miR-371 forward primer:
AAGTGCCGCCATCTTTTGAGTGT
hsa-miR-155 forward primer:
CAGTTAATGCTAATCGTGATAGGGGTT
bsa-miR-17-5p forward primer:
CAAAGTGCTTACAGTGCAGGTAG
hsa-miR-20b-5p forward primer:
CAAAGTGCTCATAGTGCAGGTAG
hsa-miR-93-5p forward primer:
CAAAGTGCTGTTCGTGCAGGTAG
hsa-miR-27b-3p forward primer:
TTCACAGTGGCTAAGTTCTGC
hsa-miR-146b forward primer:
AGTGAGAACTGAATTCCATAGGCTG
Let-7e-5p forward primer:
GCAGTGAGGTAGGAGGTTGTATAGTT
hsa-miR-15a forward primer:
CAGTAGCAGCACATAATGGTTTGTG
hsa-miR-16 forward primer:
TAGCAGCACGTAAATATTGGCG
hsa-miR-18a-3p forward primer:
ACTGCCCTAAGTGCTCCTTCTGG
hsa-miR-145 forward primer:
GTCCAGTTTTTCCCAGGAATCCCT
hsa-miR-222 forward primer:
AGCTACATCTGGCTACTGGGT
hsa-miR-218 forward primer:
GCAGTTGTGCTTGATCTAACCATGT
hsa-miR-185 forward primer:
TGGAGAGAAAGGCAGTTCCTGA
hsa-miR-151a-3p forward primer:
GCTAGACTGAAGCTCCTTGAGG
hsa-miR-140-5p forward primer:
CAGCAGTGGTTTTACCCTATGGTAG
Let-7c forward primer:
CAGTGAGGTAGTAGGTTGTATGGTT
hsa-miR-221-3p forward primer:
AGCTACATTGTCTGCTGGGTTTC
hsa-miR-21-5p forward primer:
CGACGTAGCTTATCAGACTGATGTTGA
hsa-miR-30a-5p forward primer:
CGTGTAAACATCCTCGACTGGAAG
hsa-miR-9-3p forward primer:
CGCAGATAAAGCCTAGATAACCGAAAGT
bsa-miR-325 forward primer:
CCTAGTAGGTGTCCAGTAAGTGT
hsa-miR-34a-5p forward primer:
TGGCAGTGTCTTAGCTGGTTGT
hsa-miR-422a forward primer:
ACTGGACTTAGGGTCAGAAGGC
bsa-miR-505-5p forward primer:
GGGAGCCAGGAAGTATTGATGT
hsa-miR-544a forward primer:
CGCAGATTCTGCATTTTTAGCAAGTTC
hsa-miR-363 forward primer:
CGAATTGCACGGTATCCATCTGTA
bsa-miR-487b forward primer:
GTGGTTATCCCTGTCCTGTTCG
hsa-miR-22 forward primer:
AAGCTGCCAGTTGAAGAACTGT
hsa-miR-199b-3p forward primer:
CAGACAGTAGTCTGCACATTGGTTA
bsa-miR-125a-5p forward primer:
TCCCTGAGACCCCTTTAACCTGTGA
hsa-miR-122-5p forward primer:
TGGAGTGTGACAATGGTGTTTG
hsa-miR-23a forward primer:
ATCACATTGCCAGGGATTTCC
hsa-miR-451a forward primer:
CAGAAACCGTTACCATTACTGAGTT
Let-7i-5p forward primer:
AGTGAGGTAGTAGTTTGTGCTGTT
hsa-miR-425 forward primer:
AATGACACGATCACTCCCGTTGA
hsa-miR-484 forward primer:
GCTCAGTCCCTCCCGAT

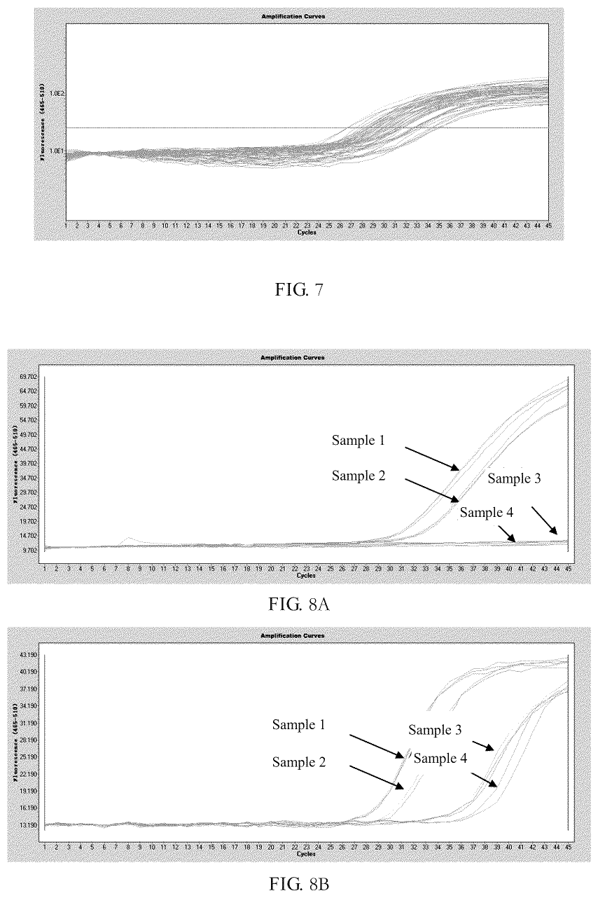

Experimental results: As shown in FIG. 7, the amplification curves of 50 microRNAs were S-shaped, and the Ct values ranged from 25 to 35. In the experiment of this method, a plasma sample size of 0.2 ml can be used for the detection of about 300-500 (species) microRNAs. Therefore, about 500-1200 kinds of microRNAs can be detected in 1 ml of peripheral blood (about 0.5 ml of plasma can be separated and obtained) using the method of the present invention. On the other hand, using the stem-loop fluorescent quantitative PCR method, the same sample size (1 ml of peripheral blood) can only detect about 40-80 (species) microRNAs.

### Example 6 The Impact of DNA Pollution on the Detection Specificity of MicroRNA Expression

PCR instrument (Bro-rad #T100), fluorescent quantitative PCR (Lightcycler480 II), tube (Axygen #PCR-0208-C), fluorescent quantitative 96-well PCR plate (Roche #047296922001), low-temperature high-speed centrifuge (Eppendorf 5424R), EP tube, pipette (Eppendorf), nematode cel-miR-39 mimic, mouse DNA sample, miR-Prep® One-step reverse transcription general kit (GeneDotech #GD-102), SYBR Green fluorescent quantitative PCR reagent (Takara #RR420A), probe method fluorescent quantitative PCR reagent (Takara #RR390A), agarose (Solarbio #A8201), SYBR safe DNA gel stain (invitrogen #S33102), DNA Marker (Takara #DL1000).

Primers and probes were synthesized and provided by Shanghai Aibosi Biotechnology Co., Ltd. The sequences thereof are the same as that in Example 1.

Experimental procedure: one-step tailing and reverse transcription are the same as those in Example 1.

Test sample:

Sample 1: cel-miR-39 mimic 1.25×10⁵ copies as standard template

Sample 2: 1.25×10⁴ copies were used as the template, 5 μl template was taken for reverse transcription according to the one-step tailing reverse transcription system disclosed in Example 1 to obtain 20 μl of cDNA product, which was then diluted.

Using mouse DNA samples as interference control:

Sample 3: mouse DNA (concentration 150 ng/μl);

Sample 4: mouse DNA sample (concentration 30 ng/μl).

IntelliMiR fluorescent probe method real-time quantitative PCR:

10 μl fluorescence quantitative reaction system includes: 2×PCR reaction solution (including Taq enzyme, dNTP mixture, MgCl₂, etc.) 5 μl, cel-miR-39 upstream amplification primer (10 μM) 0.5 μl, downstream universal primer (10 μM) 0.5 μl, miRNA cDNA gradient dilution template (10-fold diluted product) 1 μl, universal fluorescent probe (10 μM) 0.5 μl, nuclease-free water 2.5 μl.

SYBR Green real-time fluorescent quantitative PCR:

The 10 μl fluorescent quantitative reaction system includes: 2×PCR reaction solution (including Taq enzyme, SYBR Green I fluorescent dye, dNTP mixture, MgCl₂, etc.) 5 μl, cel-miR-39 upstream amplification primer (10 μM) 0.5 μl, downstream universal primer (10 μM) 0.5 μl, miRNA cDNA gradient dilution template (10-fold diluted product) 1 μl, nuclease-free water 3 μl.

Fluorescent quantitative PCR instrument reaction program: The reaction was carried out on Lightcycler480 II fluorescent quantitative PCR instrument, the reaction program was 95° C. for 30 s, 40 cycles (95° C. for 5 s, 60° C. for 30 s), and 3 replicates were set for each sample reaction.

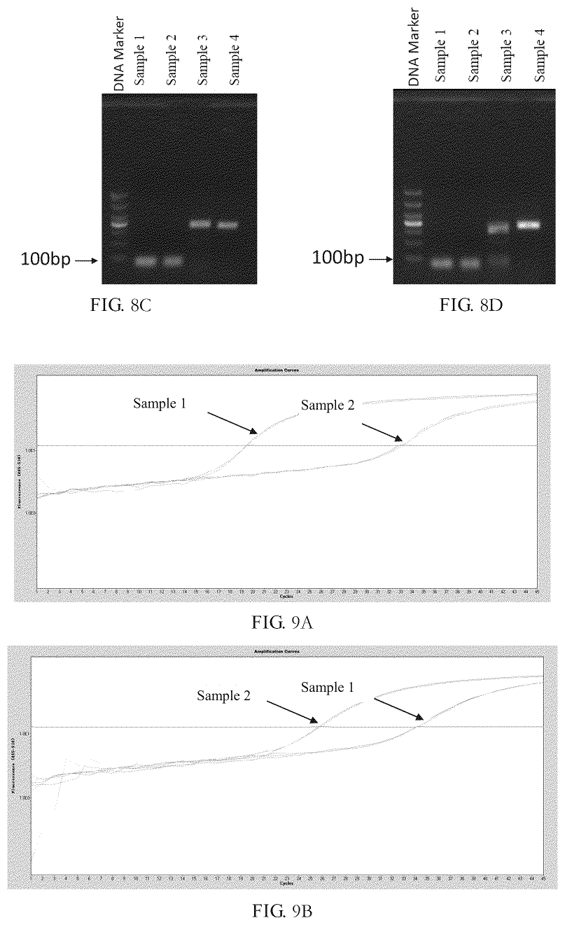

Experimental results: It is inevitable that a small amount of DNA will be mixed in the RNA sample extraction process. The existing technology mainly uses DNase to remove DNA from RNA samples, and then purifies, which will lose part of the RNA and cause unnecessary waste for RNA extraction of small or rare samples. The method for detecting the microRNA by the method of the present invention can specifically detect the microRNA and eliminate the influence of the mixed DNA during the amplification. As shown in FIG. 8A, Sample 1 and 2 detected by the fluorescent probe method of the present invention can see obvious amplification curves, while Sample 3 and 4 are mouse DNA samples, and no amplification curves can be seen. As shown in FIG. 8B, when using the SYBR Green method for the detection of microRNA, amplification curves can be seen for Sample 1 and 2, but non-specific amplification exists for interfering DNA Sample 3 and 4. Further, agarose gel electrophoresis was performed on the PCR products amplified by the two methods, and the results are shown in FIG. 8C and 8D. Both methods can generate amplified specific target bands, and the interference Sample 3 and 4 also generate obvious amplified bands. In the process of fluorescent quantitative detection, the fluorescent probe detection method can avoid the non-specific amplification signal caused by residual DNA, while the SYBR Green detection method cannot distinguish and avoid the interference signal.

### Example 7 Specificity of the Fluorescent Probe Quantitative Method of the Present Invention

PCR instrument (Bro-rad #T100), fluorescent quantitative PCR (Lightcycler480 II), tube (Axygen #PCR-0208-C), fluorescent quantitative 96-well PCR plate (Roche #047296922001), low-temperature high-speed centrifuge (Eppendorf 5424R), EP tubes, pipettes (Eppendorf), let-7a-5p and let-7c-5p mimics (Suzhou Gemma Gene Co., Ltd.), miR-Prep® One-step reverse transcription general kit (GeneDotech #GD-102, including the following components: polymerase A, reverse transcriptase MMLV, ATP, dNTP), probe method fluorescent quantitative PCR reagent (Takara #RR390A)

Primers and probes were synthesized and provided by Shanghai Aibosi Biotechnology Co., Ltd. The sequences thereof are the same as that in Example 1.

Synthesize let-7a-5p and let-7c-5p miRNA dry powder as standard. These two mimics have only one base difference:

let-7a-5p: UGAGGUAGUAGGUUGUAUAGUU
let-7c-5p: UGAGGUAGUAGGUUGUAUGGUU

Experimental procedure: one-step tailing and reverse transcription are the same as those in Example 1.

Test sample: let-7a-5p and let-7c-5p (the amount of 1 mol microRNA=6.02×10²³ copies), dilute and dissolve into a standard template of 5 fmol/μl, take 5 μl of the standard template and carry out reverse transcription according to the one-step tailing reverse transcription system in Example 1 to obtain 20 μl of cDNA product, which is then diluted to prepare for PCR reaction.

IntelliMiR fluorescence probe method real-time quantitative PCR:

Reaction system 1: 10 μl fluorescence quantitative reaction system includes: 2×PCR reaction solution (including Taq enzyme, dNTP mixture, MgCl₂, etc.) 5 μl, let-7a-5p upstream amplification primer (10 μM) 0.5 μl, downstream universal primer (10 μM) 0.5 μl, miRNA cDNA diluted template 1 μl, universal fluorescent probe (10 μM) 0.5 μl, nuclease-free water 2.5 μl.

Reaction system 2: 10 μl fluorescence quantitative reaction system includes: 2×PCR reaction solution (including Taq enzyme, dNTP mixture, MgCl₂, etc.) 5 μl, let-7c-5p upstream amplification primer (10 μM) 0.5 μl, downstream universal primer (10 μM) 0.5 μl, miRNA cDNA diluted template 1 μl, universal fluorescent probe (10 μM) 0.5 μl, nuclease-free water 2.5 μl.

Fluorescent quantitative PCR instrument reaction program: The reaction was carried out on Lightcycler480 II fluorescent quantitative PCR instrument, the reaction program was 95° C. for 30 s, 40 cycles (95° C. for 5 s, 60° C. for 30 s), and 3 replicate wells were set for each sample reaction.

The experimental results are shown in FIG. 9A. In the process of fluorescent quantitative detection using reaction system 1 (using let-7a-5p specific primers), it can be seen that the amplification curve is S-shaped, and the Ct value obtained between the same concentration target let-7a-5p and the interference let-7c-5p (there is only one base difference in sequence) differs by 11. According to the calculation of ΔCt, it means that let-7c-5p only interferes with the detection of let-7a-5p by 0.05%. As shown in FIG. 9B, in the process of fluorescent quantitative detection using reaction system 2 (using let-7c-5p specific primers), it can be seen that the amplification curve is S-shaped, and the Ct value obtained between the same concentration target let-7c-5p and the interference let-7a-5p differs by 9, and calculated according to ΔCt, it means that let-7c-5p only interferes with the detection of let-7a-5p by 0.2%.

It can be seen that the fluorescent probe detection method provided by the present invention can specifically detect the expression level of microRNAs with only one base difference.

The practice of the present invention will employ, unless otherwise indicated, conventional techniques of biotechnology, organic chemistry, inorganic chemistry, etc., and it is obvious that the invention can be carried out otherwise than as specifically described in the foregoing specification and examples. Other aspects and modifications within the scope of the invention will be apparent to those skilled in the art to which the invention pertains. Many modifications and variations are possible based on the teachings of the present invention and are therefore within the scope of the present invention. All patents, patent applications, and scientific papers mentioned herein are hereby incorporated by reference.

1. A method for detecting microRNA, comprising the steps of:
Step 1: adding the following reaction system at the same time to the test sample containing microRNA:
(a) a polyadenylic acids tailing reaction system for obtaining RNA molecules with polyadenylic acids at the 3′ end, the polyadenylic acids tailing reaction system contains: an enzyme for catalyzing a polyadenylic acids tailing reaction, and a substrate for the tailing reaction; and
(b) a reverse transcription reaction system for obtaining a reverse-transcribed cDNA product; the reverse transcription system contains: an enzyme for catalyzing the reverse transcription reaction, and a universal reverse transcription primer; wherein, the 3′ end of the universal reverse transcription primer contains a poly T oligonucleotides fragment, and the 5′ end contains an extend tag sequence fragment;
wherein the poly-T oligonucleotides fragment at the 3′ end of the universal reverse transcription primer contains about 10-20 bases, and the extended tag sequence fragment at the 5′ end contains about 20-30 bases;
Step 2: carrying out polymerase chain reaction (PCR) with the cDNA reverse transcription product obtained in Step 1 in a DNA amplification reaction and fluorescence detection system to obtain an amplification product, wherein the primer pair used in the amplification includes: a universal reverse primer and a specific forward primer specifically recognizing a target microRNA; at the same time, adding a universal fluorescent probe to the reaction system, and detecting the signal of the reporter group in the cyclic amplification step of the polymerase chain reaction, so as to obtain the detection result of the microRNA,
wherein, the universal reverse primer specifically recognizes the sequence of the universal reverse transcription primer; the specific forward primer specifically recognizes the target microRNA; and the universal fluorescent probe can specifically hybridize with the amplification product of the specific forward primer and the universal reverse primer, which is about 19-24 bases in length.

2. The method according to claim 1, wherein the 5′ end of the universal fluorescent probe has about 10-15 oligonucleotides, and the 3′ end contains a plurality of bases complementary to the extended tag sequences of the universal reverse transcription primer.

3. The method according to claim 1, wherein the Tm value of the universal reverse primer and the specific forward primer are both about 55-65° C.

4. The method according to Claim 3, wherein the Tm value of the specific forward primer and the specific forward primer are both is about 60° C.

5. The method according to claim 1, wherein the enzyme for catalyzing a polyadenylic acids tailing reaction in (a) is Escherichia coli poly (A) polymerase.

6. The method according to claim 1, wherein the enzyme for catalyzing the reverse transcription reaction in (b) is M-MLV reverse transcriptase.

7. The method according to claim 1, wherein the poly T oligonucleotides fragment at the 3′ end of the universal reverse transcription primer in (b) bas about 15 bases.

8. The method according to claim 1, wherein the extend tag sequence fragment at the 5′ end of the universal reverse transcription primer in (b) has about 27 bases.

9. The method according to claim 1, wherein the 3′ end of the universal reverse transcription primer in (b) contains an anchor base sequence, such as VN, wherein “V” represents dATP, dGTP or dCTP; “N” represents any one of dATP, dTTP, dGTP, and dCTP.

10. The method according to claim 1, wherein the universal reverse primer recognizes the extend tag sequence fragment of the universal reverse transcription primer.

11. The method according to claim 1, wherein the specific forward primer in has a sequence corresponding to the full length of the target miRNA.

12. The method according to claim 1, wherein said universal fluorescent probe recognizes and binds with the poly-T oligonucleotide fragment of the universal reverse transcription primer and one or more bases in the extend tag sequence fragment.

13. The method according to claim 12, wherein the 5′ end of the universal fluorescent probe has about 15 polyadenylic acids, and the 3′ end has a plurality of bases complementary to the extend tag sequences of the universal reverse transcription primer.

14. The method according to claim 1, wherein the 3′ labeled quencher group and the 5′ terminal labeled reporter group of the universal fluorescent probe.

15. The method according to claim 1, wherein said sample is a tissue or liquid sample containing target microRNA.

16. (canceled)

17. (canceled)

18. (canceled)

19. (canceled)

20. (canceled)

21. The method according to claim 15, wherein said sample is plasma, serum, lymph, urine, saliva, breast milk, semen, vaginal fluid, tears, spinal fluid or other body fluid sample.


SEQUENCE LISTING (7 sequences)

SEQ ID NO: 1 - DNA, 44 bp, synthetic construct
  source 1..44: mol_type = other DNA
gtccgagcagcacgatccggtgaccagtttttttttttttttvn

SEQ ID NO: 2 - DNA, 17 bp, synthetic construct
  source 1..17: mol_type = other DNA
gtccgagcagcacgatc

SEQ ID NO: 3 - DNA, 21 bp, synthetic construct
  source 1..21: mol_type = other DNA
caccgggtgtaaatcagcttg

SEQ ID NO: 4 - DNA, 19 bp, synthetic construct
  source 1..19: mol_type = other DNA
aaaaaaaaaaaaaaactgg

SEQ ID NO: 5 - DNA, 22 bp, synthetic construct
  source 1..22: mol_type = other DNA
aaaaaaaaaaaaaaactggtca

SEQ ID NO: 6 - DNA, 19 bp, synthetic construct
  source 1..19: mol_type = other DNA
aaaaaaaaaaaactggtca

SEQ ID NO: 7 - DNA, 21 bp, synthetic construct
  source 1..21: mol_type = other DNA
aaaaaaaaaaaactggtcacc